(19) United States
(12) Patent Application Publication
(10) Pub. No.: US 2021/0361405 A1
(43) Pub. Date: Nov. 25, 2021

(54) IMPLANT INSERTION DEVICE AND IMPLANT

(71) Applicant: TERUMO KABUSHIKI KAISHA, Tokyo (JP)

(72) Inventors: Hidenori TANABE, Naka-gun (JP); Manabu MIURA, Hadano-shi (JP); Yasunobu ZUSHI, Kai-shi (JP)

(73) Assignee: TERUMO KABUSHIKI KAISHA, Tokyo (JP)

(21) Appl. No.: 17/395,386

(22) Filed: Aug. 5, 2021

Related U.S. Application Data
(63) Continuation of application No. PCT/JP2020/003775, filed on Jan. 31, 2020.

(30) Foreign Application Priority Data
Feb. 6, 2019 (JP) 2019-019456

(51) Int. Cl. A61F 2/00; A61B 17/34

(52) U.S. Cl. CPC A61F 2/0095; A61B 2017/3456; A61B 17/3421; A61B 17/3468

(57) ABSTRACT

An implant insertion device includes: a tubular body having a distal portion that is insertable into subcutaneous tissue and an insertion hole that extends proximally from the distal portion in an axial direction; an elongated implant configured to promote tissue regeneration by attachment of cells; and an elongated body that is accommodated in the insertion hole of the tubular body and is configured to move along the insertion hole of the tubular body and to protrude from the distal portion of the tubular body. The elongated body is configured to advance with respect to the tubular body and thereby push a distal portion of the implant in a distal direction from the insertion hole of the tubular body.

### CROSS-REFERENCE TO RELATED APPLICATIONS

This application is a bypass continuation of PCT Application No. PCT/JP2020/003775, filed on Jan. 31, 2020, which claims priority to Japanese Application No. 2019-019456, filed on Feb. 6, 2019. The contents of these applications are hereby incorporated by reference in their entireties.

### BACKGROUND

The present disclosure relates to an implant insertion device, which punctures skin and places an implant to be implanted in a body, and the implant.

In order to treat a tissue of which function has deteriorated, various treatment methods for installing an implant that promotes tissue regeneration at such a site have been proposed. For example, there is proposed a method for implanting a porous collagen fiber, which has numerous pores formed inside, in a damaged site (JP 2012-500203 A). If such a collagen fiber is implanted, there is an effect of gathering cells around the fiber and promoting regeneration of a damaged tissue.

### SUMMARY

In general, the implant is implanted by a method of incising a skin tissue to expose the damaged tissue and fixing the collagen fiber to be implanted to the damaged tissue with a ligature clip or a tissue adhesive. However, such a method is highly invasive and places a great burden on a patient, and thus, there is a demand for a method that is less burdensome.

Therefore, an object of the present disclosure is to provide an implant insertion device suitable for inserting an implant having an ability to promote tissue regeneration in a damaged tissue, as well as the implant itself.

According to one embodiment, an implant insertion device includes: a tubular body having a distal portion that is insertable into a subcutaneous tissue and an insertion hole that penetrates in an axial direction and extends from the distal portion to a proximal side; an elongated implant having an ability to promote tissue regeneration with attachment of a cell; and an elongated body that is accommodated in the insertion hole of the tubular body and is capable of moving along the insertion hole of the tubular body and protruding from the distal portion of the tubular body. The elongated body pushes a distal portion of the implant in a distal direction from the insertion hole of the tubular body as advancing with respect to the tubular body.

According to another embodiment, an implant used in an implant insertion device includes distal portion provided with an attachment portion that can attach to an implantation site by being separated from a distal portion of the elongated body.

According to the implant insertion device of the present invention, the implant can be transferred to an implantation site while being accommodated in the elongated body.

### BRIEF DESCRIPTION OF DRAWINGS

FIG. 1 is a cross-sectional view of an implant insertion device according to a first embodiment of the present invention;

FIG. 2A is an enlarged cross-sectional view in a state in which an implant is fixed by a fixing mechanism of FIG. 1, and FIG. 2B is an enlarged cross-sectional view of the fixing mechanism of FIG. 1 in the middle of detaching the implant;

FIG. 3 is an explanatory view illustrating how the implant is implanted in a damaged tissue with the implant insertion device of FIG. 1;

FIG. 4 is a cross-sectional view of a state in which a skin is punctured with the implant insertion device of FIG. 1;

FIG. 5 is a cross-sectional view of the implant insertion device of FIG. 1 in a state in which an elongated body protrudes;

FIG. 6 is a cross-sectional view of a state in which the implant is being pulled out after being transferred to an implantation site by the implant insertion device of FIG. 1;

FIG. 7 is an enlarged cross-sectional view of a vicinity of an elongated body hub of an implant insertion device according to a first modification of the first embodiment;

FIG. 8A is an enlarged cross-sectional view illustrating an implant insertion device according to a second modification of the first embodiment in a state in which a cap member fixes an implant, and FIG. 8B is an enlarged cross-sectional view illustrating a state in which the cap member of FIG. 8A has detached the implant;

FIG. 9 is a cross-sectional view of an implant insertion device according to a second embodiment;

FIG. 10 is a cross-sectional view illustrating a second position of an elongated body of the implant insertion device of FIG. 9;

FIG. 11 is a cross-sectional view illustrating a first position of the elongated body of the implant insertion device of FIG. 9;

FIG. 12 is a cross-sectional view of a state in which an implant is being pulled out after being transferred to an implantation site by the implant insertion device of FIG. 9;

FIG. 13 is an enlarged cross-sectional view of a vicinity of a distal end of an implant insertion device according to a third embodiment;

FIG. 14 is an enlarged cross-sectional view of a vicinity of a distal end of an implant insertion device according to a fourth embodiment;

FIG. 15 is a cross-sectional view of an implant insertion device according to a fifth embodiment;

FIG. 16A is an enlarged perspective view in a fixed state of a fixing mechanism of the implant insertion device of FIG. 15, FIG. 16B is an enlarged perspective view in a semi-released state of the same fixing mechanism, and FIG. 16C is an enlarged perspective view in a released state of the same fixing mechanism;

FIG. 17 is a cross-sectional view of an implant insertion device according to a sixth embodiment;

FIG. 18A is an enlarged cross-sectional view in a fixed state of a fixing mechanism of the implant insertion device of FIG. 17, and FIG. 18B is an enlarged cross-sectional view in a released state of the fixing mechanism of the implant insertion device of FIG. 17;

FIG. 19 is a cross-sectional view of an implant insertion device according to a seventh embodiment;

FIG. 20A is a view illustrating a first example of a pattern of an uneven portion of the implant insertion device according to the seventh embodiment, FIG. 20B is a view illustrating a second example of the pattern of the uneven portion of the implant insertion device according to the seventh embodiment, and FIG. 20C is a view illustrating a third example of the pattern of the uneven portion of the implant insertion device according to the seventh embodiment; and

FIG. 21A is a cross-sectional view of an implant insertion device according to a first modification of the seventh embodiment, and FIG. 21B is a cross-sectional view of an implant insertion device according to a second modification of the seventh embodiment.

### DETAILED DESCRIPTION

Hereinafter, preferred embodiments of the present invention will be described in detail with reference to the accompanying drawings.

### First Embodiment

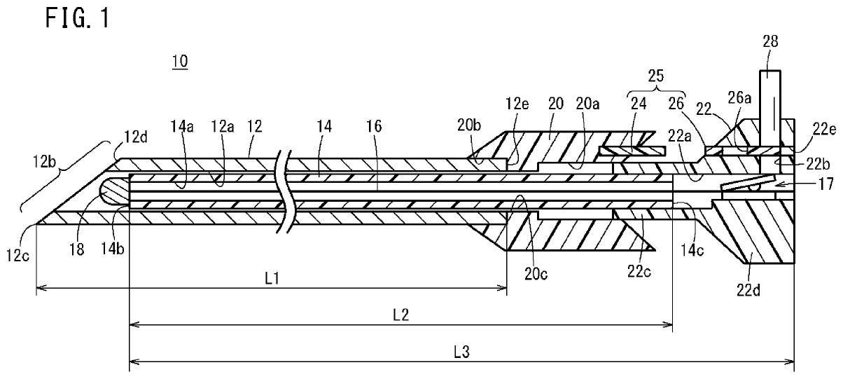

An implant insertion device 10 according to the present embodiment is used when implanting an implant in an implantation site having a damaged tissue. As illustrated in FIG. 1, the implant insertion device 10 includes: a tubular body 12 having a sharp distal portion 12b; an elongated body 14 arranged inside the tubular body 12; and an implant 16 accommodated in the elongated body 14.

The tubular body 12 is a substantially cylindrical member in which an insertion hole 12a extending in the axial direction is formed, and is formed to be elongated in the axial direction. The tubular body 12 is made of metal such as stainless steel, and the distal portion 12b is formed with a distal surface 12d inclined with respect to the axial direction. A sharp needle tip 12c is formed at an end on the distal side of the distal surface 12d so as to puncture a skin S or a subcutaneous tissue.

The tubular body 12 is formed to have an outer diameter of, for example, about 1.1 to 1.3 mm, and an inner diameter of the internal insertion hole 12a is formed to be, for example, about 0.8 to 1.0 mm. A length L1 from the needle tip 12c at the distal end of the tubular body 12 to a proximal portion 12e of the tubular body 12 can be set to, for example, about 100 mm.

A tubular body hub 20 configure to operate the tubular body 12 is connected to the proximal side of the tubular body 12. The tubular body hub 20 is made of, for example, resin, and is formed to have a larger outer diameter than the tubular body 12 in order to facilitate the operation of the tubular body 12. Inside the tubular body hub 20, a tubular body mounting portion 20b, an insertion hole 20c, and an accommodating portion 20a are provided in order from the distal side in the axial direction.

The tubular body mounting portion 20b is an axially extending hole, formed to have an inner diameter substantially the same as the outer diameter of the tubular body 12, and accommodates and holds a vicinity of the proximal portion 12e of the tubular body 12. The tubular body 12 is joined to the tubular body hub 20 by an adhesive or the like at the tubular body mounting portion 20b. The insertion hole 20c is an axially extending hole formed to have substantially the same inner diameter as the insertion hole 12a of the tubular body 12, and is configured such that the elongated body 14 can be inserted therein. The accommodating portion 20a is an axially extending hole, and is configured to be capable of accommodating a sliding portion 22c on the distal side of the elongated body hub 22 that will be described below. The sliding portion 22c moves inside the accommodating portion 20a while sliding on an inner surface of the accommodating portion 20a. Further, a release pin 24 is formed on the proximal side of the tubular body hub 20 so as to extend to the proximal side.

The elongated body 14 is a substantially cylindrical member in which an implant accommodating hole 14a extending in the axial direction is formed, and is formed to be elongated in the axial direction. The elongated body 14 is made of metal such as stainless steel. A distal portion 14b of the elongated body 14 is formed of a distal surface substantially perpendicular to the axial direction and is formed to be blunt. The elongated body 14 is configured to be slidable in the axial direction along the insertion hole 12a of the tubular body 12. Further, the elongated body 14 is formed to be longer than the tubular body 12, and the distal portion 14b can protrude to the distal side of the needle tip 12c of the tubular body 12.

The elongated body 14 is formed to have an outer diameter of, for example, about 0.8 mm, and an inner diameter of the implant accommodating hole 14a is formed to be, for example, about 0.6 mm. A length L2 from the distal portion 14b to a proximal portion 14c of the elongated body 14 can be set to, for example, about 150 mm.

The elongated body hub 22 configured to operate the elongated body 14 is provided on the proximal side of the elongated body 14. The elongated body hub 22 includes an insertion hole 22a that accommodates and holds a vicinity of the proximal portion 14c of the elongated body 14. That is, the elongated body hub 22 and the elongated body 14 are integrally joined at the insertion hole 22a. The distal side of the elongated body hub 22 is provided with the cylindrical sliding portion 22c axially extending to the distal side, and a hub main body 22d is provided on the proximal side of the sliding portion 22c. The hub main body 22d is formed to have a larger diameter than the sliding portion 22c in order to facilitate the operation of the elongated body 14. A length L3 from the distal portion 14b of the elongated body 14 to a proximal portion of the elongated body hub 22 can be set to, for example, about 200 mm.

The insertion hole 22a of the elongated body hub 22 is formed so as to penetrate the inside of the elongated body hub 22 in the axial direction, and a fixing mechanism 17 is provided inside the insertion hole 22a. The fixing mechanism 17 is configured to fix the implant 16 to the elongated body 14 by fixing the proximal side of the implant 16. The fixing mechanism 17 of the present embodiment is formed in a clip shape that sandwiches and holds the implant 16 by a biasing force of a spring (not illustrated).

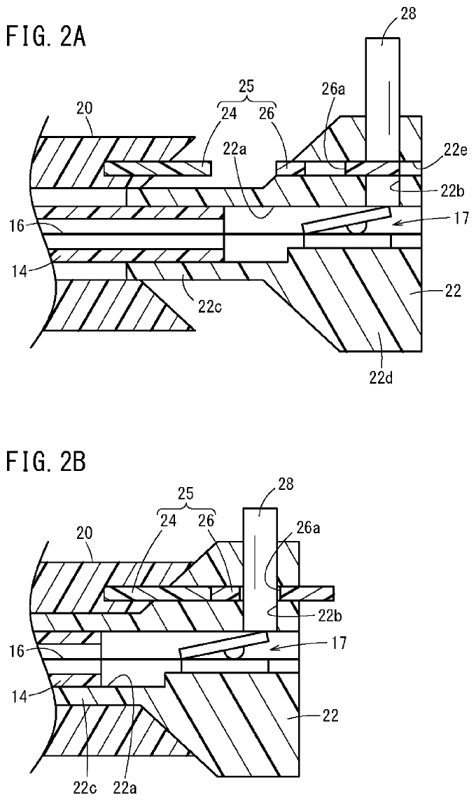

As illustrated in FIG. 2A, the hub main body 22d is formed with an operation hole 22b extending in the radial direction. A detachment operation pin 28 is inserted into the operation hole 22b. When the detachment operation pin 28 is pushed radially inward (lower side of FIG. 2A), the fixation of the implant 16 by the fixing mechanism 17 is released.

The tubular body hub 20 and the elongated body hub 22 are provided with a locking mechanism 25 in order to prevent the fixing of the implant 16 from being released at an unintended timing. The locking mechanism 25 includes the release pin 24 extending from the tubular body hub 20 to the proximal side, and a locking plate 26 provided on the elongated body hub 22. Between these, the locking plate 26 is configured to be movable in the axial direction along a guide hole 22e provided in the hub main body 22d. The locking plate 26 is provided with an opening 26a that can communicate with the operation hole 22b. As illustrated in FIG. 2A, the locking plate 26 is arranged at a position that closes the operation hole 22b in the initial state. Further, when the elongated body hub 22 is pushed into the tubular body hub 20 as illustrated in FIG. 2B, the locking plate 26 is displaced to the proximal side by the release pin 24, and the opening 26a of the locking plate 26 communicates with the operation hole 22b. As a result, the locking mechanism 25 is unlocked, and the fixing mechanism 17 can be pressed by the detachment operation pin 28. That is, the implant 16 can be released by the fixing mechanism 17 by causing the elongated body hub 22 to protrude to the distal side.

As illustrated in FIG. 1, the implant 16 is accommodated in the implant accommodating hole 14a of the elongated body 14. The implant 16 is a fibrous member that is elongated in the axial direction, and is formed of a member in which cells that regenerate tissue easily gather when being implanted in a damaged tissue in a living body. Examples of such a member include a porous collagen fiber in which numerous micropores are formed in a cross section. As the implant 16, for example, a thread-like one having an average diameter of about 0.4 mm and a length of 150 mm or more can be used. An outer diameter of the implant 16 is smaller than the inner diameter of the implant accommodating hole 14a of the elongated body 14, and the implant 16 is accommodated in the implant accommodating hole 14a in a state in which tension is not applied to the implant 16.

An attachment portion 18, formed to have a larger diameter than a main body of the implant 16, is attached to the distal side of the implant 16. The attachment portion 18 is formed to have a larger diameter than the implant accommodating hole 14a of the elongated body 14. Therefore, the attachment portion 18 can be referred to as an enlarged-diameter portion. The elongated portion 18 is locked to the distal portion 14b of the elongated body 14, and has a function as a cover of the elongated body 14. Therefore, the attachment portion 18 can be referred to as a cover portion. Further, when the attachment portion 18 is separated from the elongated body 14 in living tissue, the attachment portion 18 exerts a function of engaging in the living tissue and attachment the implant 16 to a target site.

The proximal side of the implant 16 extends from the elongated body 14 to the proximal side and is sandwiched by the fixing mechanism 17. That is, the implant 16 is fixed to the elongated body 14 by the fixing mechanism 17.

The implant insertion device 10 according to the present embodiment is configured as described above, and its operational effect will be described hereinafter.

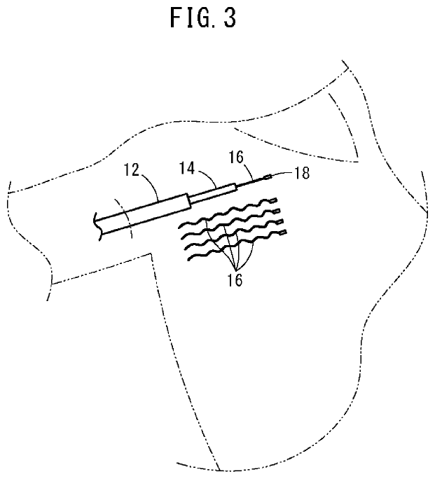

As illustrated in FIG. 3, the implant insertion device 10 can be used in a procedure of inserting the implant 16 in a vicinity of a lymph node where a damaged lymphatic vessel is located. The lymphatic vessel is damaged by lymphadenectomy or radiation treatment during tumor treatment. When the lymphatic vessel is damaged and causes dysfunction, an edema sometimes occurs because a lymphatic fluid does not flow normally. When implanting the implant 16 made of the collagen fiber in order to improve such dysfunction, cell proliferation and organization can be induced to promote regeneration of the lymphatic vessel. As illustrated in the drawing, a plurality of the implants 16 are implanted in parallel to more reliably regenerate the lymphatic vessel. Hereinafter, an operation of the implant insertion device 10 will be described.

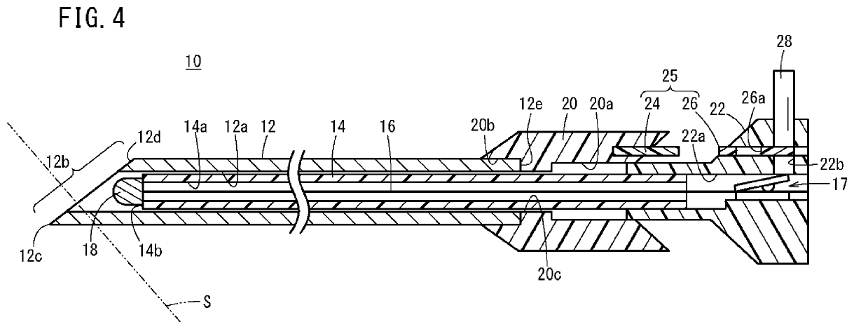

First, the skin S near an implantation site is punctured with the implant insertion device 10 as illustrated in FIG. 4. The puncturing is performed in a state in which the elongated body 14 is pulled into the insertion hole 12a of the tubular body 12. The sharp needle tip 12c of the tubular body 12 makes it easy to puncture the skin S. Thereafter, the tubular body 12 is advanced in a subcutaneous tissue from a punctured site toward the implantation site. The operation of advancing the tubular body 12 can be performed by the tubular body hub 20.

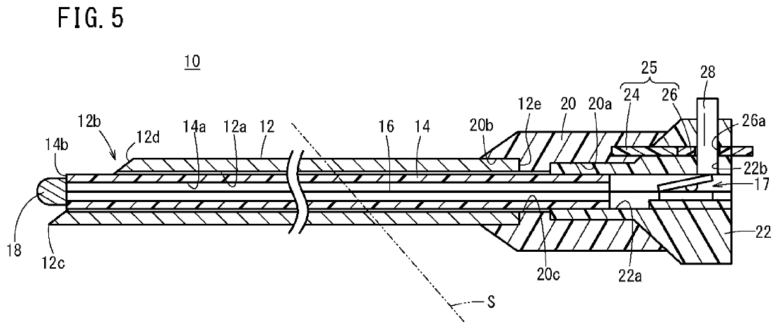

Next, when the tubular body 12 reaches a predetermined site near the implantation site as illustrated in FIG. 5, the elongated body hub 22 is pushed to cause the elongated body 14 to protrude from the distal end of the tubular body 12 and advance the elongated body 14 to the implantation site. Since the distal portion 14b of the elongated body 14 is formed to be blunt, damage in a vicinity of the implantation site can be suppressed. The implant 16 is transferred to the implantation site in the state of being covered with the elongated body 14. At that time, a load such as a pulling force is not applied on the implant 16, and thus, it is possible to prevent breakage of a micro-tissue of the implant 16 and deterioration of the cell attachment property.

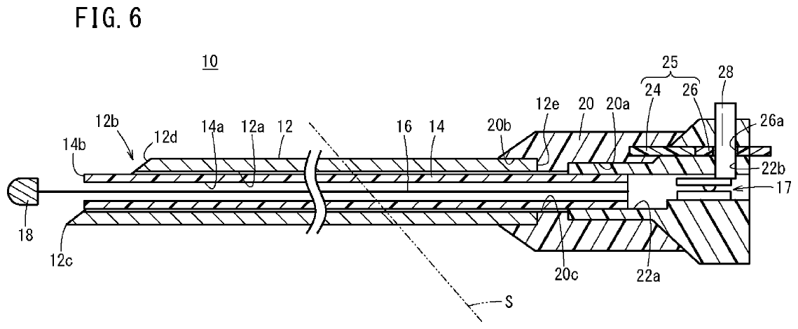

Thereafter, as illustrated in FIG. 6, the fixation of the implant 16 by the fixing mechanism 17 is released by pushing the detachment operation pin 28, and an operation of pulling out the implant insertion device 10 including the elongated body 14 and the tubular body 12 is performed. At that time, the attachment portion 18 at the distal end of the implant 16 is kept in the state of being engaged with the implantation site by being pressed against the subcutaneous tissue. When the elongated body 14 and the tubular body 12 are pulled out while leaving the implant 16, the implant 16 can be implanted with a minimum load without applying the pulling force on the implant 16. As a result, the implant 16 can be implanted in a target tissue while maintaining the micro-tissue of the implant 16. The insertion of the implant 16 is completed as above.

The implant insertion device 10 and the implant 16 of the present embodiment have the following effects.

The above-described implant insertion device 10 includes: the tubular body 12 having the distal portion 12b that can be inserted into the subcutaneous tissue and the insertion hole 12a that penetrates in the axial direction and extends from the distal portion 12b to the proximal side; the elongated implant 16 having an ability to promote tissue regeneration by attachment of cells; and the elongated body 14 that is accommodated in the insertion hole 12a of the tubular body 12 and can move along the insertion hole 12a of the tubular body 12 and protrude from the distal portion 12b of the tubular body 12. The elongated body 14 is configured to push the distal portion of the implant 16 in the distal direction from the insertion hole 12a of the tubular body 12 as advancing with respect to the tubular body 12. As a result, the implant 16 can be transferred to the implantation site while being accommodated in the elongated body 14, and it is possible to prevent a load such as the pulling force from being applied to the implant 16. As a result, the micro-tissue of the implant 16 can be protected and the cell attachment property can be improved.

In the above-described implant insertion device 10, the fixing mechanism 17 that detachably fixes the implant 16 to the elongated body 14 may be provided. As a result, the implant 16 can be removed from the elongated body 14 and implanted at a desired implantation site.

In the above-described implant insertion device 10, the distal portion 12b of the tubular body 12 is formed to be sharp, and the distal portion 14b of the elongated body 14 is formed to be blunt. As a result, damage to a blood vessel, a nerve, and the like can be prevented when the distal portion 14b of the elongated body 14 is caused to protrude.

In the above-described implant insertion device 10, the distal portion of the implant 16 may be provided with the attachment portion 18 that can be attached to the implantation site by being separated from the distal portion 14b of the elongated body 14. As a result, the displacement of the implant 16 can be prevented when the tubular body 12 and the elongated body 14 are pulled out, and the implant 16 can be reliably implanted at the implantation site.

In the above-described implant insertion device 10, the attachment portion 18 may be formed to have a larger diameter than the main body of the implant 16. As a result, the attachment portion 18 can be reliably attached to the implantation site.

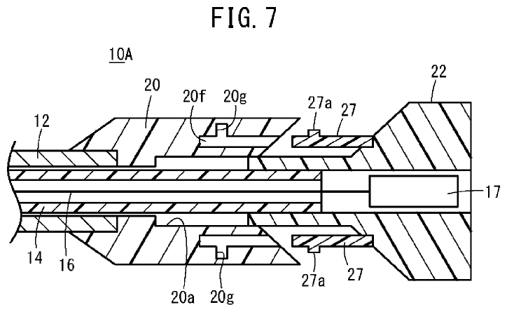

The implant insertion device 10 according to the present embodiment is not limited to the above-described embodiment. For example, as illustrated in an implant insertion device 10A of a first modification of FIG. 7, the elongated body hub 22 may be provided with an elongated body fixing piece 27 that fixes the elongated body hub 22 to the tubular body hub 20. The elongated body fixing piece 27 has a protrusion 27a that extends in the axial direction and protrudes in the width direction. The tubular body hub 20 of a portion corresponding to the elongated body fixing piece 27 is provided with a hole 20f to accommodate the elongated body fixing piece 27.

A recess 20g that engages with the protrusion 27a is provided on a side portion of the hole 20f. In the implant insertion device 10A, when the elongated body hub 22 is pushed into the tubular body hub 20, the protrusion 27a of the elongated body fixing piece 27 engages with the recess 20g of the hole 20f, so that the elongated body hub 22 is displaced with respect to the tubular body hub 20. As a result, it is possible to prevent unintentionally movement of the elongated body hub 22 and deviation of the implantation site when the fixing of the implant 16 is released.

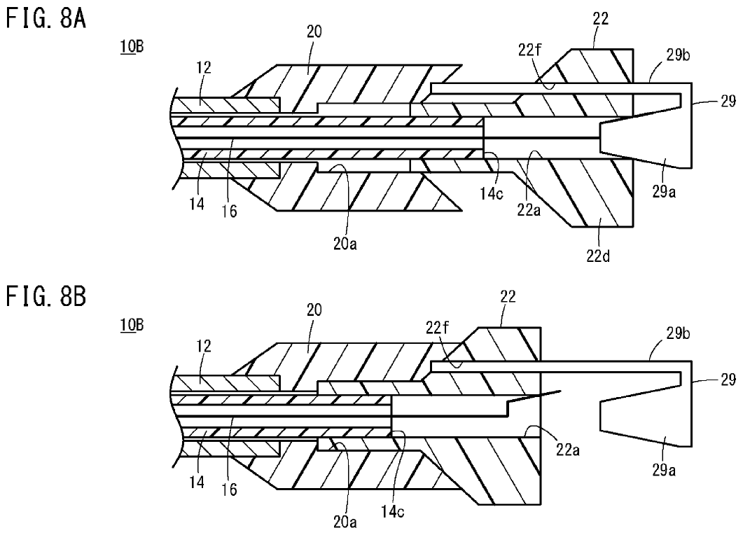

As illustrated in FIG. 8A, an implant insertion device 10B of a second modification has a different structure of the fixing mechanism 17. That is, the implant insertion device 10B includes a cap member 29 instead of the fixing mechanism 17 having the clip shape.

The cap member 29 includes a cap portion 29a inserted into the insertion hole 22a of the elongated body hub 22, and a release piece 29b bent and extended from the cap portion 29a. The release piece 29b penetrates the hole 22f of the elongated body hub 22 and extends to the distal side of the hub main body 22d. The proximal side of the implant 16 is sandwiched and fixed by the cap portion 29a.

When the elongated body hub 22 is pushed into the tubular body hub 20 as illustrated in FIG. 8B, the release piece 29b abuts on the tubular body hub 20 and stops, so that the cap portion 29a comes off from the insertion hole 22a of the elongated body hub 22. As a result, the fixing of the implant 16 is released.

### Second Embodiment

There is a case where the implant 16 needs to pass through a hard fibrous tissue when being transferred to a damaged tissue. Further, there is also a case where it is desirable to make the implant 16 to pass a vicinity of a tissue such as a blood vessel, a nerve, and a human body that is not desired to be damaged while passing through a subcutaneous tissue. Therefore, an implant insertion device 30 capable of flexibly switching sharpness and bluntness of a distal portion according to a site and transferring the implant 16 to an implantation site will be described in the present embodiment. Note that, in the implant insertion device 30, the same configurations as those in the implant insertion device 10 of FIG. 1 are denoted by the same reference signs, and detailed descriptions thereof will be omitted.

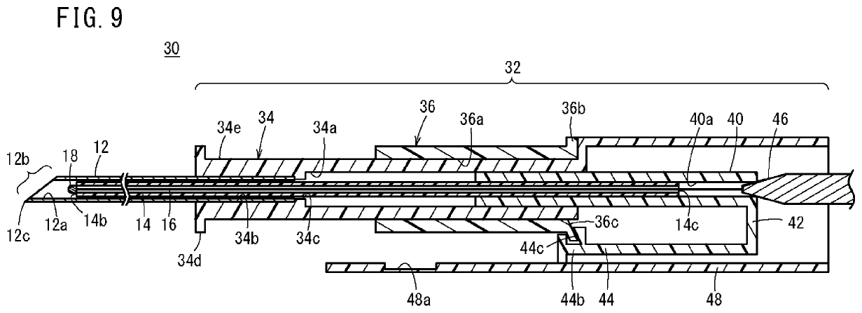

As illustrated in FIG. 9, the implant insertion device 30 of the present embodiment includes a moving mechanism 32 that freely displaces the elongated body 14 in the axial direction in addition to the tubular body 12, the elongated body 14, and the implant 16.

The moving mechanism 32 includes: a tubular body hub 34 joined to the proximal side of the tubular body 12; an elongated body hub 40 joined to the proximal side of the elongated body 14; and a slider 36 provided so as to be slidable in the axial direction with respect to the tubular body hub 34. Among these, the tubular body hub 34 includes a sliding portion 34e formed on the distal side and a handle portion 48 formed on the proximal side and having a larger diameter than the sliding portion 34e. The handle portion 48 is a site that is gripped by a user's hand to operate the tubular body 12, and is formed in an outer shape that is easy to grip. The elongated body hub 40 is accommodated inside the handle portion 48.

The sliding portion 34e is a site formed in a cylindrical shape, and is a portion on which the slider 36 mounted on the outer peripheral portion thereof slides. A stopper 34d is provided at a distal end of the sliding portion 34e to prevent the slider 36 from falling off. Inside the sliding portion 34e, a tubular body mounting portion 34b, an insertion hole 34c, and an accommodating portion 34a are formed as holes penetrating in the axial direction in order from the distal side in the axial direction. The proximal side of the tubular body 12 is mounted and joined to the tubular body mounting portion 34b. The insertion hole 34c is the hole in which the elongated body 14 is freely inserted. A main body of the elongated body hub 40 is accommodated in the accommodating portion 34a. In the present embodiment, the elongated body hub 40 is configured to be displaced while sliding inside the accommodating portion 34a.

A through-hole 40a penetrating in the axial direction is provided inside the elongated body hub 40. The through-hole 40a is formed to have substantially the same inner diameter as an outer diameter of the elongated body 14, and accommodates and holds the proximal side of the elongated body 14. Further, a cap 46 is mounted to the proximal side of the through-hole 40a. The cap 46 sandwiches and holds the proximal side of the implant 16, which extends from the proximal portion 14c of the elongated body 14, with the through-hole 40a. That is, the cap 46 forms a fixing mechanism that fixes the implant 16 to the elongated body 14 in the present embodiment. Note that the cap 46 is formed to be elongated in the axial direction and protrude from the proximal side of the handle portion 48 so as to be easily removable.

An arm portion 42 extends radially outward from the proximal side of the elongated body hub 40. An engaging portion 44 extends to the distal side in the axial direction from an outer end of the arm portion 42. The engaging portion 44 includes a claw portion 44b that engages with a flange portion 36b of the slider 36. The engaging portion 44 is arranged to release an engagement with the flange portion 36b in a natural state in which there is no stress, but is blocked by an inner wall of the handle portion 48 to remain in a position of engaging with the flange portion 36b. The flange portion 36b that engages with the claw portion 44b has an inclined portion 36c that is inclined to the radially outer side of the flange portion 36b and to the proximal side. The claw portion 44b has an inclined portion 44c that is inclined to the radially inner side and to the distal side so as to engage with the inclined portion 36c of the flange portion 36b.

A recess 48a formed in a concave shape is formed on an inner peripheral surface side of the claw portion 44b of the handle portion 48 in a portion in a movement direction. When the slider 36 and the engaging portion 44 are advanced, a free end side of the engaging portion 44 enters the recess 48a, and the claw portion 44b is caught by the recess 48a. Further, when the slider 36 is retracted to the proximal side, the claw portion 44b and the flange portion 36b engage with each other so that the elongated body 14 is accommodated inside the tubular body 12. When the inclined portion 44c of the claw portion 44b abuts on the inclined portion 36c of the flange portion 36b, the arm portion 42 is pulled up inward. As a result, the engagement between a lower protruding portion of the claw portion 44b and the recess 48a is released.

The slider 36 is a member formed in a substantially cylindrical shape, and is configured such that an inner peripheral surface 36a thereof can be displaced in the axial direction while sliding on the sliding portion 34e. The flange portion 36b having a large diameter is provided in a proximal portion of the slider 36. The flange portion 36b is engaged with the claw portion 44b of the engaging portion 44 extending from the elongated body hub 40. That is, the elongated body hub 40 is configured to be displaced in the axial direction by displacing the slider 36 in the axial direction. The slider 36 can be operated with an index finger when the handle portion 48 is grasped by a hand, and is configured such that an axial position of the distal portion 14b of the elongated body 14 can be easily changed. When the slider 36 is advanced, the claw portion 44b of the elongated body hub 40 is also caught by the recess 48a in a state in which the claw portion 44b of the elongated body hub 40 is caught by the flange portion 36b, and thus, locking is doubly performed by catching at the two sites such that the elongated body 14 is not unintentionally retracted.

The implant insertion device 30 of the present embodiment is configured as described above, and its operational effect will be described hereinafter.

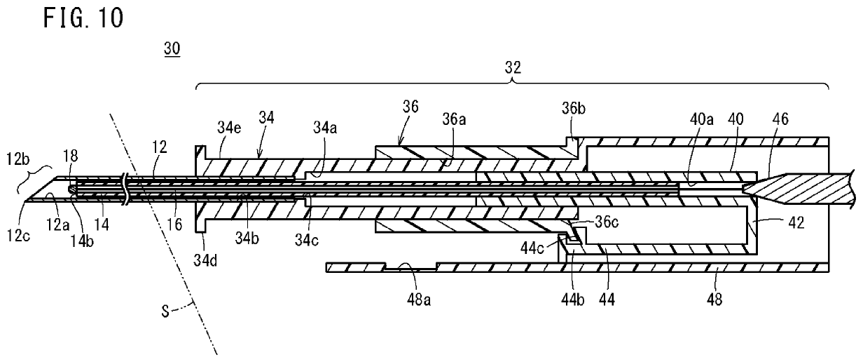

First, the skin S near an implantation site is punctured with the tubular body 12 of the implant insertion device 30 as illustrated in FIG. 10. In this case, the distal portion 14b of the elongated body 14 is set in a state of being located at a second position on the proximal side of the distal portion 12b of the tubular body 12. As a result, the sharp needle tip 12c of the tubular body 12 is not obstructed by the distal portion 14b of the elongated body 14 formed to be blunt, and the skin S can be easily punctured.

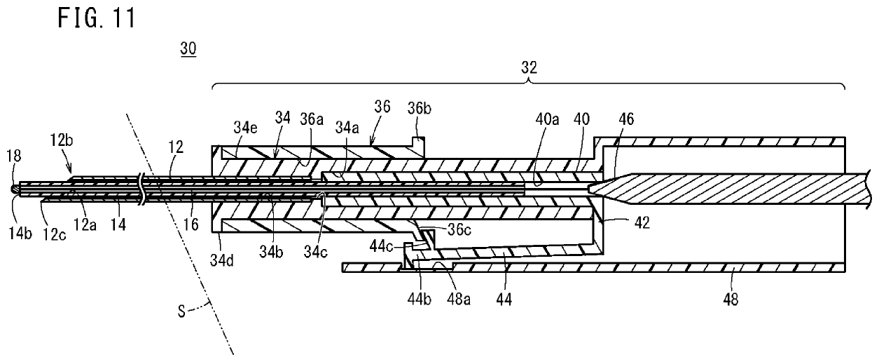

Next, the elongated body 14 is advanced inside a skin tissue toward the implantation site. In this case, the moving mechanism 32 is operated to displace the distal portion 14b of the elongated body 14 to a first position to protrude from the distal end of the tubular body 12 as illustrated in FIG. 11 according to a passage site. The bluntly formed distal portion 14b of the elongated body 14 and attachment portion 18 protrude at the first position, and thus, can pass without damaging a blood vessel, a nerve, and a ligament.

Further, the slider 36 of the moving mechanism 32 is moved to the proximal side to displace the distal portion 14b of the elongated body 14 to the second position on the proximal side of the tubular body 12 from the distal portion 12b as illustrated in FIG. 10 when passage of a hard fibrous tissue is required in the middle of advancing toward the implantation site. As a result, the needle tip 12c of the tubular body 12 can puncture and pass the hard fibrous tissue.

As described above, the implant insertion device 30 is moved to the implantation site while displacing the elongated body 14 to the second position in FIG. 10 and the first position in FIG. 11.

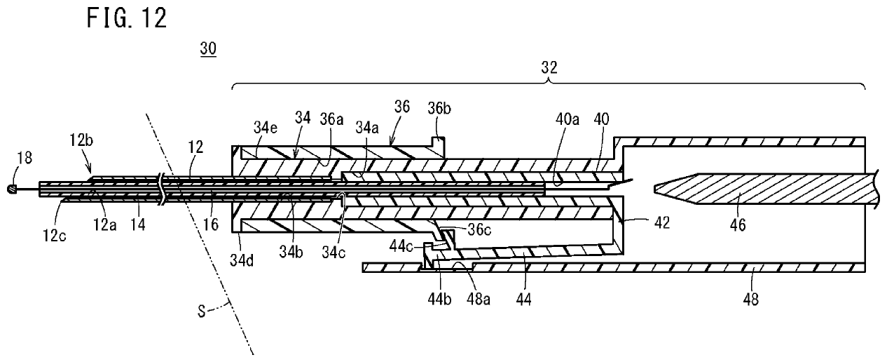

Thereafter, the distal portion 14b of the elongated body 14 is arranged at the implantation site of the implant 16. Then, the cap 46 is removed from the elongated body hub 40 to release the fixing of the implant 16 as illustrated in FIG. 12. Next, an operation of pulling the tubular body hub 34 to the proximal side to pull out the implant insertion device 30 from the implantation site is performed. As a result, the insertion of the implant 16 is completed, and the implant 16 is implanted at the predetermined implantation site.

The implant insertion device 30 of the present embodiment has the following effects.

The implant insertion device 30 includes, on the proximal portion side of the tubular body 12 and the elongated body 14, the moving mechanism 32 that displaces the elongated body 14 in the axial direction. The moving mechanism 32 can be freely displaced to the first position at which the distal portion 14b of the elongated body 14 protrudes from the distal portion 12b of the tubular body 12, and the second position at which the distal portion 14b of the elongated body 14 is retracted to the proximal side of the distal portion 12b of the tubular body 12. As a result, the implant insertion device 30 can be safely advanced while passing through the subcutaneous tissue without damaging the blood vessel, nerve, ligament, and the like.

### Third Embodiment

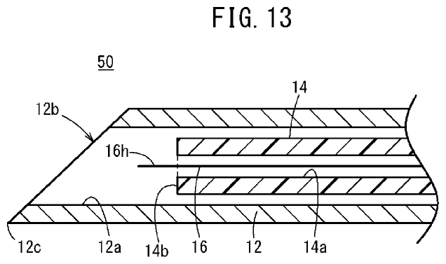

As illustrated in FIG. 13, in an implant insertion device 50 of the present embodiment, the attachment portion 18 is not provided at a distal end 16h of the implant 16 accommodated in the elongated body 14, and the distal end 16h of the implant 16 protrudes from the distal portion 14b of the elongated body 14 by a predetermined length. Other configurations are the same as those of the implant insertion device 10 in FIG. 1.

Even with the implant insertion device 50 of the present embodiment, the implant 16 can be transferred to an implantation site in a state of being covered with the elongated body 14. Further, the implant 16 can be implanted at the implantation site by pulling out the tubular body 12 and the elongated body 14 although the attachment portion 18 is not provided.

### Fourth Embodiment

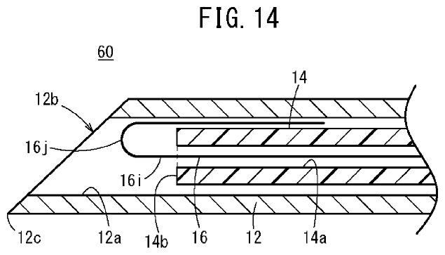

As illustrated in FIG. 14, in an implant insertion device 60 of the present embodiment, the attachment portion 18 is not provided at a distal portion 16i of the implant 16 accommodated in the elongated body 14, and the distal portion 16i of the implant 16 protrudes from the distal portion 14b of the elongated body 14 by a predetermined length. In the present embodiment, the distal portion 16i of the implant 16 is folded to the proximal side at a folded portion 16j, and is arranged along a side surface of the elongated body 14. Note that other configurations are the same as those of the implant insertion device 10 in FIG. 1.

Even with the implant insertion device 60 of the present embodiment, most of the implant 16 can be transferred to an implantation site in a state of being covered with the elongated body 14. Further, the implant 16 can be implanted at the implantation site by pulling out the tubular body 12 and the elongated body 14 although the attachment portion 18 is not provided.

### Fifth Embodiment

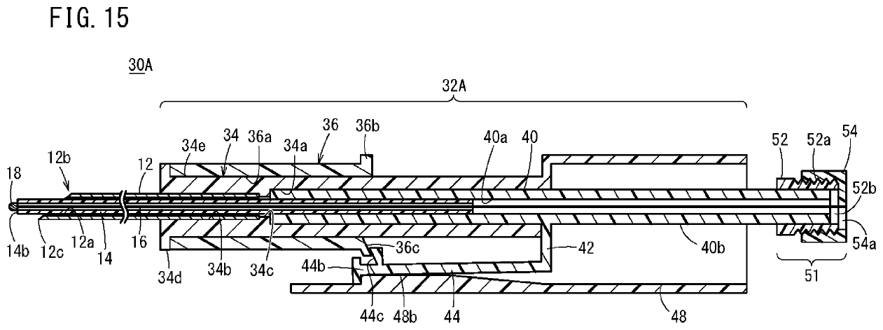

As illustrated in FIG. 15, in an implant insertion device 30A of the present embodiment, the elongated body hub 40 protrudes and extends to the proximal side of the handle portion 48, and a fixing mechanism 51 that detachably fixes the implant 16 is provided in a proximal portion of the elongated body hub 40. Note that, in the implant insertion device 30A of the present embodiment, the same configurations as those of the implant insertion device 30 described with reference to FIGS. 9 to 12 are denoted by the same reference signs, and detailed descriptions thereof will be omitted.

As illustrated in FIG. 15, in the elongated body hub 40 of the present embodiment, the arm portion 42 extends radially outward from a side portion near the middle in the axial direction, and the engaging portion 44 extends from the arm portion 42 to the distal side in the axial direction. The claw portion 44b engaging with the flange portion 36b of the slider 36 is provided at a distal end of the engaging portion 44.

An inner wall of the handle portion 48 of the present embodiment is provided with an engaging protrusion 48b, which biases the engaging portion 44 toward the axis, in a portion facing the engaging portion 44. The engaging protrusion 48b is formed by an inclination that gradually protrudes inward from the proximal side to the distal side, and an end on the distal side is formed to be substantially perpendicular to the axial direction. When the slider 36 is moved to the distal side to move the claw portion 44b to the distal side of the engaging protrusion 48b, the engaging portion 44 comes off from the engaging protrusion 48b, and the engagement between the claw portion 44b and the flange portion 36b is released. As a result, only the elongated body 14 can be advanced. Note that, when the slider 36 is pulled to the proximal side, the claw portion 44b and the flange portion 36b are engaged again, and the elongated body 14 can be accommodated in the tubular body 12.

The elongated body hub 40 includes an extending portion 40b extending to the proximal side of the arm portion 42. The extending portion 40b is formed to have such a length that the proximal side protrudes from the handle portion 48 even in a state in which the elongated body hub 40 is advanced with respect to the handle portion 48, and is exposed to the outside of the handle portion 48. The through-hole 40a is also formed inside the extending portion 40b, and the implant 16 is arranged in the through-hole 40a. Further, the fixing mechanism 51 that detachably fixes the implant 16 is attached to a proximal portion of the extending portion 40b.

The fixing mechanism 51 includes a fitting member 52 mounted to the proximal portion of the extending portion 40b from the outside, and a cap member 54 mounted to the fitting member 52. The fitting member 52 is formed in a cylindrical shape and is joined to an outer peripheral portion of the extending portion 40b. A screw mechanism 52a is provided on an outer peripheral portion of the fitting member 52, and the cap member 54 is screwed and mounted from the outer peripheral side of the fitting member 52 via the screw mechanism 52a.

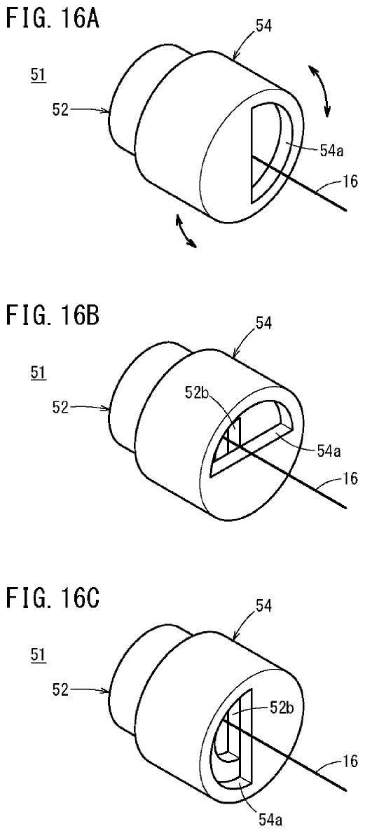

As illustrated in FIG. 16A, the cap member 54 is rotatable about an axis along the screw mechanism 52a as indicated by the arrow in the drawing. As illustrated in FIGS. 16B and 16C, a semicircular opening 52b is formed in a proximal portion of the fitting member 52 on the inner side. Further, a semicircular opening 54a is formed in a proximal portion of the cap member 54. The implant 16 is arranged to pass through these openings 52b and 54a.

As illustrated in FIG. 16A, when the opening 52b of the fitting member 52 and the opening 54a of the cap member 54 are located not to communicate with each other, the implant 16 is sandwiched between the fitting member 52 and the cap member 54 and fixed to the elongated body hub 40. That is, the implant 16 is set in the state of being fixed with respect to the elongated body 14.

As illustrated in FIG. 16B, the fixing of the implant 16 is in a semi-released state in a state in which a part of the opening 52b of the fitting member 52 and a part of the opening 54a of the cap member 54 communicate with each other. In the semi-released state, the implant 16 can move in the axial direction while receiving frictional resistance from the openings 52b and 54a. Furthermore, when rotated as illustrated in FIG. 16C, the opening 52b of the fitting member 52 and the opening 54a of the cap member 54 communicate with each other, and the implant 16 is completely released from the fixed state. In this case, the implant 16 can be inserted without receiving the resistance of the openings 52b and 54a, and the implant insertion device 30A can be pulled out while leaving the implant 16 at an implantation site.

The implant insertion device 30A of the present embodiment has the following effects.

The implant insertion device 30A includes the elongated body hub 40 that holds the elongated body 14 and accommodates the implant 16 in the internal through-hole 40a, the handle portion 48 that accommodates the elongated body hub 40 to be displaceable in the axial direction, the slider 36 that is mounted to the handle portion 48 to be slidable in the axial direction, and the engaging portion 44 that engages the slider 36 and the elongated body hub 40 with each other as the moving mechanism 32A that displaces the elongated body 14 in the axial direction. The elongated body hub 40 extends to the proximal side of the handle portion 48, and the fixing mechanism 51 that detachably fixes the implant 16 is provided in the proximal portion of the elongated body hub 40. As a result, the fixed state of the implant 16 can be released by operating the fixing mechanism 51 on the proximal side of the handle portion 48.

In the above-described implant insertion device 30A, the fixing mechanism 51 is formed of the fitting member 52 that is mounted to the proximal portion of the elongated body hub 40 and has the opening 52b that allows passage of the implant 16 and the cap member 54 that is rotatably mounted to the fitting member 52 and has the opening 54a that can communicate with the opening 52b when being rotated. As a result, the openings 52b and 54a can be made to communicate with each other or closed by rotating the cap member 54, so that the fixed state and the released state of the implant 16 can be switched.

In the above-described implant insertion device 30A, the engaging portion 44 may be provided with the engaging protrusion 48b, which is formed in a shape in which the engagement with the slider 36 is released in a state in which no stress is applied and deforms the engaging portion 44 in the direction of engaging with the slider 36, on an inner peripheral surface of the handle portion 48. In this case, the engaging protrusion 48b may be configured to release the engagement between the engaging portion 44 and the slider 36 when the slider 36 is displaced to the distal end. With this configuration, it is possible to prevent a problem that a position of the elongated body 14 is inadvertently changed in the protruding state of the elongated body 14.

### Sixth Embodiment

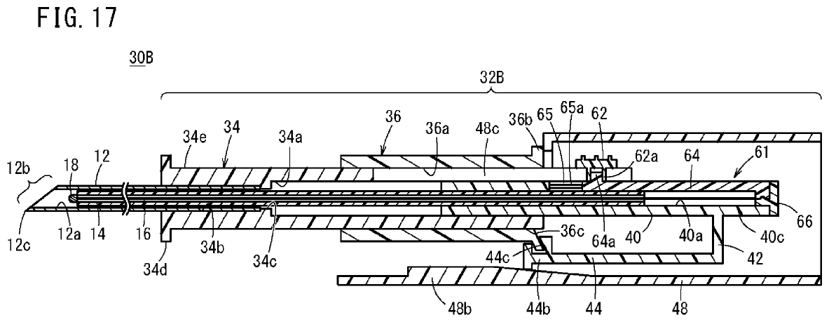

As illustrated in FIG. 17, an implant insertion device 30B of the present embodiment is different from the implant insertion device 30A in FIG. 15 in terms of a moving mechanism 32B provided with a fixing mechanism 61 that detachably fixes the implant 16. Other points are the same as those of the implant insertion device 30A. In the implant insertion device 30B, the same configurations as those in the implant insertion device 30A are denoted by the same reference signs, and detailed descriptions thereof will be omitted.

In the moving mechanism 32B of the implant insertion device 30B, the elongated body hub 40 includes an extending portion 40c protruding from the arm portion 42 and the engaging portion 44 to the proximal side. The extending portion 40c is formed in a tubular shape having the through-hole 40a inside. The extending portion 40c of the present embodiment is formed to be shorter than the extending portion 40b of FIG. 15, and is formed to have a length that does not protrude from the handle portion 48.

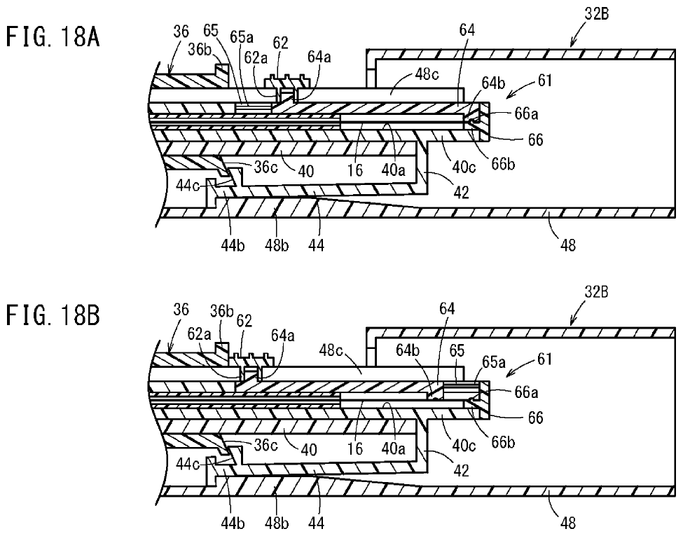

The fixing mechanism 61 is provided in a vicinity of a proximal portion of the elongated body hub 40 including the extending portion 40c. The fixing mechanism 61 of the present embodiment includes a release knob 62, a locking piece 64, and an end cap 66. Among these, the end cap 66 is inserted and mounted to the through-hole 40a that is open in the proximal portion of the elongated body hub 40 as illustrated in FIG. 18A. The end cap 66 includes an insertion portion 66b that protrudes to the distal side in the axial direction in a shape that closes the lower half of a cross section of the through-hole 40a. As the insertion portion 66b is inserted into the through-hole 40a, the end cap 66 is mounted to the proximal portion of the elongated body hub 40.

Further, a convex portion 66a is provided at an upper end of the insertion portion 66b of the end cap 66. The convex portion 66a protrudes from the upper end of the insertion portion 66b, and the implant 16 is arranged so as to pass the upper side of the convex portion 66a.

The elongated body hub 40 is provided with a notch 65 for accommodating the locking piece 64 so as to be slidable in the axial direction over a predetermined range from the proximal portion thereof, and the locking piece 64 is arranged in the notch 65. A guide rail mechanism 65a is provided on a side portion of the notch 65, and the locking piece 64 is guided by the guide rail mechanism 65a and slides in the axial direction. The locking piece 64 is a rod-shaped member that is elongated in the axial direction, and a proximal-side protrusion 64b that can be engaged with the convex portion 66a of the end cap 66 is provided on the proximal side thereof. As illustrated in FIG. 17, when the locking piece 64 is displaced to the proximal side, the proximal-side protrusion 64b engages with the convex portion 66a, thereby sandwiching and fixing the implant 16. Further, a distal-side protrusion 64a that engages with the release knob 62 is formed so as to protrude outward on the distal side of the locking piece 64. A connection portion 62a of the release knob 62 is engaged with the distal-side protrusion 64a. That is, the locking piece 64 is configured to be displaced in the axial direction integrally with the release knob 62.

The release knob 62 is mounted to the handle portion 48 in a state in which the handle portion 48 can be displaced in the axial direction along a guide portion 48c formed by cutting out the handle portion 48 in a groove shape in the axial direction. When the release knob 62 is displaced toward the distal side in the axial direction, the locking piece 64 moves to the distal side, and the engagement state between the proximal-side protrusion 64b of the locking piece 64 and the convex portion 66a of the end cap 66 is released, so that the fixing of the implant 16 is released.

Hereinafter, an operational effect of the implant insertion device 30B of the present embodiment will be described.

As illustrated in FIG. 17, a subcutaneous tissue of a living body is punctured in a state in which the slider 36 is pulled to the proximal side. When the tubular body 12 reaches a target position, the slider 36 is moved to the distal side to make the elongated body 14 protrude from the tubular body 12. As a result, the locking piece 64 and the release knob 62 also move to the distal side as the elongated body hub 40 moves forward as illustrated in FIG. 18A. Then, the release knob 62 is exposed to the outside of the handle portion 48 and can be operated.

Thereafter, the release knob 62 is moved in the distal direction. As a result, as illustrated in FIG. 18B, the locking piece 64 moves to the distal side, and the proximal-side protrusion 64b of the locking piece 64 comes off from the convex portion 66a of the end cap 66, so that the fixed state of the implant 16 is released. Thereafter, the implant 16 is implanted by pulling out the implant insertion device 30B.

The implant insertion device 30B of the present embodiment has the following effects.

The implant insertion device 30B includes the fixing mechanism 61 having the locking piece 64 that is accommodated in the elongated body hub 40 to be movable in the axial direction, the end cap 66 that is mounted to the proximal side of the elongated body hub 40 and holds the implant 16 by engaging with the locking piece 64, and the release knob 62 that engages with the locking piece 64 and displaces the locking piece 64 to the distal side to release the engagement state between the locking piece 64 and the end cap 66. With such a configuration, the fixed state of the implant 16 can be easily released by operating the release knob 62, and the operability is improved.

### Seventh Embodiment

In the case of using the implant insertion devices 10, 10A, 10B, 30, 30A, and 30B described in the above embodiments, it is used by accurately positioning the target site while checking the positions of the tubular body 12, the elongated body 14 and the attachment portion 18 using an ultrasonic diagnostic imaging apparatus. Therefore, in the present embodiment, a description will be given regarding an example in which processing for enhancement of a reflectance to ultrasonic waves is performed such that positions of distal portions of the tubular body 12, the elongated body 14, and the attachment portion 18 are more clearly reflected by the ultrasonic diagnostic imaging apparatus. Note that, in an implant insertion device described in the present embodiment, the same constituent members as those described with reference to FIGS. 1 to 18B are denoted by the same reference signs, and detailed descriptions thereof will be omitted.

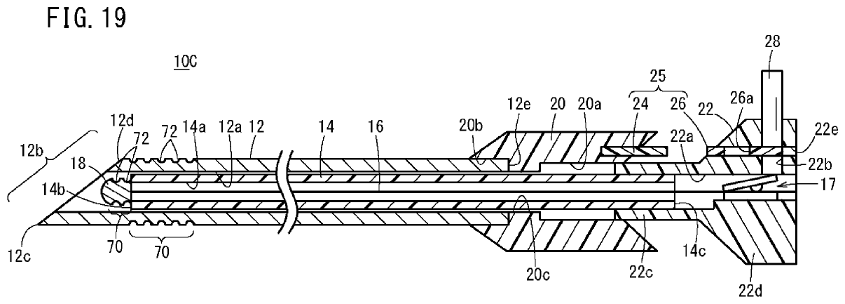

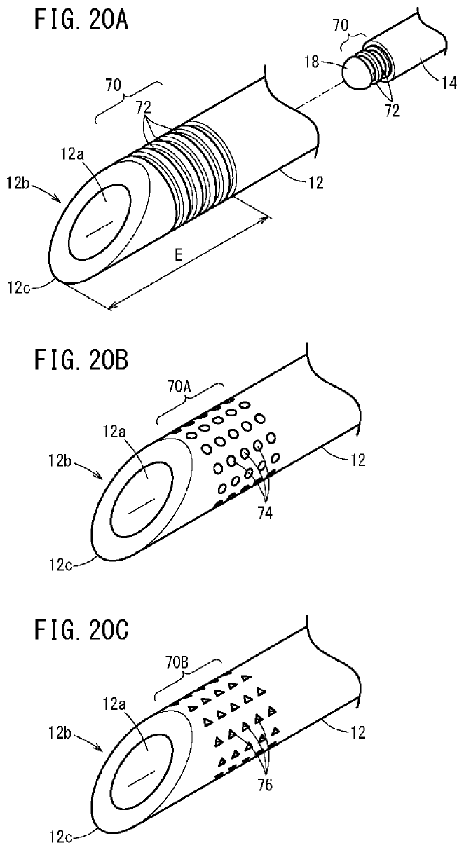

In an implant insertion device 10C illustrated in FIG. 19, uneven portions 70 are provided on an outer peripheral portion of the tubular body 12 in a vicinity of the distal portion 12b and an outer peripheral portion of the attachment portion 18. As illustrated in FIG. 20A, the uneven portion 70 is formed of a plurality of grooves 72, and the grooves 72 are formed in an annular shape over the entire circumference of the outer peripheral portion of the tubular body 12. Note that the groove 72 may be formed of one or a plurality of grooves formed in a spiral shape. In the tubular body 12, a range E for forming the uneven portion 70 is preferably set to at least 1 to 3 cm from the needle tip 12c of the distal portion 12b. Further, the groove 72 is also formed in the outer peripheral portion of the attachment portion 18 at a distal end of the elongated body 14 as illustrated in the partially enlarged view of FIG. 20A.

With this configuration, the tubular body 12 and the attachment portion 18 can be clearly visible using the ultrasonic diagnostic imaging apparatus, and the implant 16 can be accurately arranged at the target site.

Note that the same effect can be obtained by forming an uneven portion 70A provided with a plurality of dimples 74 recessed in a spherical shape as illustrated in FIG. 20B, instead of the uneven portion 70 formed of the grooves 72 in FIG. 20A. Further, the same effect can be obtained by providing uneven portions 70B each having a plurality of wedge-shaped recesses 76 in the tubular body 12 and the attachment portion 18 as illustrated in FIG. 20C, instead of the uneven portion 70 of FIG. 20A.

The uneven portion 70 of the present embodiment can be provided at the same sites in the implant insertion device 30 described with reference to FIGS. 9 to 12. Further, the uneven portion 70 of the present embodiment may be provided in the implant insertion device 50 described with reference to FIG. 13 or the implant insertion device 60 described with reference to FIG. 14.

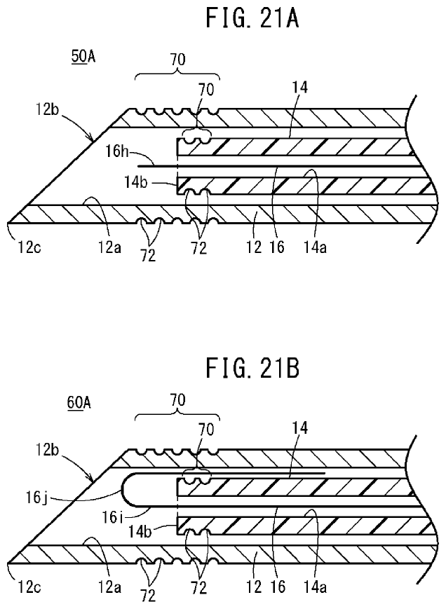

An implant insertion device 50A according to a first modification illustrated in FIG. 21A is obtained by providing the uneven portion 70 in the implant insertion device 50 in FIG. 13. Note that in the implant insertion device 50A of FIG. 21A, the same configurations as those of the implant insertion device 50 of FIG. 13 are denoted by the same reference signs, and detailed descriptions thereof will be omitted. As illustrated in FIG. 21A, the uneven portion 70 is provided on an outer peripheral portion of the tubular body 12 in a vicinity of the needle tip 12c in the implant insertion device 50A. Further, the uneven portion 70 is also provided on an outer peripheral portion in a vicinity of the distal portion 14b of the elongated body 14 in the implant insertion device 50A.

With the above configuration, a position of a distal end of the tubular body 12 can be clearly visible by an ultrasonic diagnostic imaging apparatus at the time of using the implant insertion device 50A. Further, since the uneven portion 70 is provided in the vicinity of the distal portion 14b of the elongated body 14, a position of a distal end of the elongated body 14 can be clearly visible by the ultrasonic diagnostic imaging apparatus when the elongated body 14 is made to protrude from the tubular body 12.

An implant insertion device 60A according to a second modification illustrated in FIG. 21B is obtained by providing the uneven portion 70 in the implant insertion device 60 in FIG. 14. The uneven portions 70 are provided on an outer peripheral portion in a vicinity of the distal portion 12b of the tubular body 12 of the implant insertion device 60A and an outer peripheral portion in a vicinity of the distal portion 14b of the elongated body 14. Even in the second modification, a position of a distal end of the tubular body 12 and a position of a distal end of the elongated body 14 can be clearly visible by an ultrasonic diagnostic imaging apparatus at the time of using the implant insertion device 60A. As a result, the implant 16 can be accurately placed at a target position.

Although the present invention has been described with the preferred embodiments as above, it is obvious that the present invention is not limited to the above-described embodiments, and various modifications can be made within a scope not departing from a gist of the present invention.

What is claimed is:

1. An implant insertion device comprising:
a tubular body having a distal portion that is insertable into subcutaneous tissue and an insertion hole that extends proximally from the distal portion in an axial direction;
an elongated implant configured to promote tissue regeneration by attachment of cells; and
an elongated body that is accommodated in the insertion hole of the tubular body and is configured to move along the insertion hole of the tubular body and to protrude from the distal portion of the tubular body,
wherein the elongated body is configured to advance with respect to the tubular body and thereby push a distal portion of the implant in a distal direction from the insertion hole of the tubular body.

2. The implant insertion device according to claim 1, further comprising a fixing mechanism configured to detachably fix the implant to the elongated body.

3. The implant insertion device according to claim 1, wherein the distal portion of the tubular body is sharp, and a distal portion of the elongated body is blunt.

4. The implant insertion device according to claim 3, further comprising:
a moving mechanism that is located at a proximal portion of the tubular body and the elongated body and that is configured to displace the elongated body in the axial direction,
wherein the moving mechanism is freely displaceable to a first position at which the distal portion of the elongated body protrudes from the distal portion of the tubular body and a second position at which the distal portion of the elongated body is retracted to a proximal side of the distal portion of the tubular body.

5. The implant insertion device according to claim 1, wherein the distal portion of the implant comprises an attachment portion that is configured to separate from the distal portion of the elongated body and to attach to an implantation site.

6. The implant insertion device according to claim 5, wherein a diameter of the attachment portion is larger than a diameter of a main body of the implant.

7. The implant insertion device according to claim 1, wherein the implant is accommodated in the elongated body in a state in which a distal side of the implant protrudes distally from the distal portion of the elongated body.

8. The implant insertion device according to claim 1, wherein the implant protrudes distally from the distal portion of the elongated body, and the distal portion of the implant is folded to the proximal side and extends along a side surface of the elongated body.

9. An elongated implant for use with an implant insertion device comprising: a tubular body having a distal portion that is insertable into subcutaneous tissue and an insertion hole that extends proximally from the distal portion in an axial direction, and an elongated body that is accommodated in the insertion hole of the tubular body and is configured to move along the insertion hole of the tubular body and to protrude from the distal portion of the tubular body, wherein the elongated body comprises an implant accommodating hole and is configured to advance with respect to the tubular body and thereby push a distal portion of the implant in a distal direction from the insertion hole of the tubular body, the elongated implant comprising:
a main body configured to be accommodated in the implant accommodating hole; and
an attachment portion located at a distal end of the main body, wherein a diameter of the attachment portion is greater than a diameter of the main body, and wherein the attachment portion is configured to separate from the distal portion of the elongated body and to attach to an implantation site,
wherein the elongated implant is configured to promote tissue regeneration by attachment of cells.

10. The implant according to claim 9, wherein a diameter of the attachment portion larger than a diameter of the implant accommodating hole.

11. A method for inserting an elongated implant, the method comprising:
providing an implant insertion device comprising:
a tubular body having a distal portion that is insertable into subcutaneous tissue and an insertion hole that extends proximally from the distal portion in an axial direction,
an elongated implant configured to promote tissue regeneration by attachment of cells, and
an elongated body that is accommodated in the insertion hole of the tubular body and is configured to move along the insertion hole of the tubular body and to protrude from the distal portion of the tubular body; and
advancing the elongated body with respect to the tubular body such that the elongated body pushes a distal portion of the implant in a distal direction from the insertion hole of the tubular body.
